(19) United States
(12) Patent Application Publication
(10) Pub. No.: US 2020/0064422 A1
(43) Pub. Date: Feb. 27, 2020

(54) ACOUSTIC GENERATOR FOR MRI DEVICES, AND MRI DEVICE PROVIDED WITH SAME

(71) Applicant: Hitachi, Ltd., Tokyo (JP)

(72) Inventor: Takeshi YATSUO, Tokyo (JP)

(73) Assignee: Hitachi, Ltd., Tokyo (JP)

(21) Appl. No.: 16/084,035

(22) PCT Filed: Feb. 7, 2017
(86) PCT No.: PCT/JP2017/004342

§ 371 Date: Sep. 11, 2018

(30) Foreign Application Priority Data
Mar. 29, 2016 (JP) 2016-065239

(51) Int. Cl. G01R 33/28; A61B 5/055; H04R 1/02; H04R 9/06; H04R 9/02; H04R 7/04; H04R 7/18; G01R 33/34

(52) U.S. Cl. CPC G01R 33/283; A61B 5/055; H04R 1/028; G01R 33/34; H04R 9/025; H04R 7/04; H04R 7/18; H04R 9/06

(57) ABSTRACT

The present invention provides an acoustic generator for an MRI device which is good for communication with a subject and an MRI device having the acoustic generator. An acoustic generator for an MRI device has an acoustic coil, a diaphragm which oscillates based on force generated in the acoustic coil by an interaction between current flowing in the acoustic coil and a magnetic field for imaging generated by the MRI device, and a supporting member oscillatably supporting the diaphragm. A force generated in the acoustic coil changes according to a change in the current flowing in the acoustic coil. The diaphragm vibrates air in accordance with the change in the force, thereby generating acoustics.

### TECHNICAL FIELD

The present invention relates to a nuclear magnetic resonance imaging (hereinbelow, called “MRI”) device of measuring a nuclear magnetic resonance (hereinbelow, called “NMR”) signal from hydrogen, phosphorus, or the like in a subject and forming an image of a nuclear density distribution, a relaxation time distribution, and the like or an acoustic generator used for the MRI device.

### BACKGROUND ART

An MRI device is a device measuring NMR signals generated by atomic nucleus spins constructing a tissue in a subject, particularly, a human body and two-dimensionally or three-dimensionally imaging the form or function of the head, abdomen, extremities, or the like of the subject. In imaging, a phase encode which varies according to a gradient magnetic field is given to an NMR signal, frequency encoding is performed, and the resultant signal is measured as time-series data. By two-dimensional or three-dimensional Fourier transform, the measured NMR signal is reconstructed as an image.

The NMR signal measured is very weak. When an RF pulse is superimposed, fake occurs in an image. Consequently, measurement is performed in a shield room which is electromagnetically shielded and a subject to be imaged and an operator performing an operation on the outside of the shield room are physically isolated. However, communication using a microphone and a speaker is performed for an instruction of holding breath at the time of performing imaging when the motion of the body is stopped, announcement when any inconvenience occurs, and other various necessities. In Patent Literature 1 to be described below, a system for the communication using a microphone and a speaker is disclosed.

As described above, Patent Literature 1 discloses a communication system using a microphone and a speaker for communication between a subject and an operator of an MRI device. However, a concrete structure of the speaker is not described.

### CITATION LIST

### Patent Literature

PTL 1: Japanese Patent Application Laid-Open No. 2002-102203

### SUMMARY OF INVENTION

### Technical Problem

A speaker generally used is configured by a voice coil, a diaphragm, and a permanent magnet. Current from an amplifier flows in the voice coil, and the diaphragm integrated with the voice coil is driven by the interphase action with a magnetic flux of the permanent magnet to generate a sound. It is difficult to use a speaker having such a structure near an MRI device having a permanent magnet or a superconducting magnet. For example, the speaker has to be placed in a position apart from a magnetic field generation source in a shield room. It is consequently unsuitable to use the speaker as means for communication to a subject.

As a speaker which can be disposed near a subject of an MRI device, a speaker using a piezoelectric element is considered. A speaker using a piezoelectric element has a structure of generating a sound by using a mechanical displacement of the piezoelectric element which occurs when voltage is applied to the piezoelectric element. However, a mechanical displacement amount of the piezoelectric element when a voltage is applied is small, so that it is difficult to obtain a proper volume. Consequently, there is no speaker which is good for communication with a subject of an MRI device, and an effort for the communication is being made in an insufficient state.

An object of the present invention is to provide an acoustic generator for an MRI device, which is good for communication with a subject of an MRI device and an MRI device having the acoustic generator.

### Solution to Problem

An acoustic generator for an MRI device, which solves the problem has: an acoustic coil provided in a magnetic field for imaging generated by a magnetostatic field generation coil or a gradient magnetic field generation coil of an MRI device or in a magnetic field for imaging generated by both of the magnetostatic field generation coil and the gradient magnetic field generation coil, separately from the magnetostatic field generation coil and the gradient magnetic field generation coil; a diaphragm which oscillates on the basis of a force generated by the acoustic coil by action between current flowing in the acoustic coil and the magnetic field for imaging generated by the MRI device; and a supporting member supporting the diaphragm so as to be oscillatable, and is characterized in that the current flowing in the acoustic coil is a current whose value changes to generate acoustics, the force generated in the acoustic coil changes according to a change in the current, and the diaphragm vibrates air in accordance with the change in the force, thereby generating acoustics.

### Advantageous Effects of Invention

According to the present invention, an acoustic generator for an MRI device, which is good for communication with a subject of an MRI device, and an MRI device having the acoustic generator can be obtained.

### BRIEF DESCRIPTION OF DRAWINGS

FIG. 1 is an explanatory diagram explaining an example of an MRI device to which the present invention is applied.

FIG. 2 is an explanatory diagram explaining an example of an acoustic generator for the MRI device to which the present invention is applied.

FIG. 3 is an explanatory diagram explaining operation of the acoustic generator for the MRI device to which the present invention is applied.

FIG. 4 is an explanatory diagram explaining the relations between currents and displacements of a diaphragm in a section in an XZ plane illustrated in FIG. 3.

FIG. 5 is an explanatory diagram explaining the relations between currents and displacements of a diaphragm in the section in the XZ plane illustrated in FIG. 3.

FIG. 6 is an explanatory diagram explaining another embodiment of the acoustic generator for the MRI device illustrated in FIG. 2.

FIG. 7 is an explanatory diagram explaining another embodiment of the acoustic generator for the MRI device illustrated in FIG. 2.

FIG. 8 is an explanatory diagram explaining operation of the embodiment illustrated in FIG. 7.

FIG. 9 is an explanatory diagram explaining further another embodiment of the acoustic generator for the MRI device illustrated in FIG. 2.

FIG. 10 is an explanatory diagram explaining further another embodiment of the acoustic generator for the MRI device illustrated in FIG. 2.

FIG. 11 is an explanatory diagram explaining the shape of an acoustic coil for examining generation strength of magnetic fields.

FIG. 12 is an explanatory diagram explaining magnetic field intensities generated by the acoustic coil illustrated in FIG. 11.

FIG. 13 is an explanatory diagram explaining the shape of an acoustic coil having a figure-of-eight shape for examining the generation intensities of magnetic fields.

FIG. 14 is an explanatory diagram explaining magnetic field intensities generated by the acoustic coil having the figure-of-eight shape illustrated in FIG. 13.

FIG. 15 is an explanatory diagram illustrating an embodiment of a conductor pattern of an acoustic coil to which the present invention is applied.

FIG. 16 is an explanatory diagram explaining installation of an acoustic generator for an MRI device in an MRI device using a cylindrical magnet.

FIG. 17 is an explanatory diagram explaining installation of an acoustic generator for an MRI device in an MRI device of a perpendicular magnetic field type.

FIG. 18 is an explanatory diagram illustrating an embodiment of installing an acoustic generator for an MRI device using a blast pipe for cooling.

FIG. 19 is an explanatory diagram explaining an embodiment of installing an acoustic generator for an MRI device in an MRI device using a cylindrical magnet to execute active noise cancellation.

FIG. 20 is an explanatory diagram explaining an embodiment of installing an acoustic generator for an MRI device in a top board of a bed to execute active noise cancellation.

### DESCRIPTION OF EMBODIMENTS

### 1. Introduction

Next, an embodiment of the present invention will be described with reference to the drawings. Components to which the same reference numeral are designated in the drawings referred to have similar operations, and similar effects are produced. To avoid repetition of the description, the description of the operations and effects related to components of the same reference numeral will not be repeated. The following embodiment solves the problem of the above-described invention and produces the effect of the above-described invention and, in addition, solves a problem other than the problem of the above-described invention and also produces an effect other than the above-described effect. The solutions to the problem and the effects will be mentioned in the description of the embodiments.

2. An Example of MRI Device to which The Present Invention is Applied

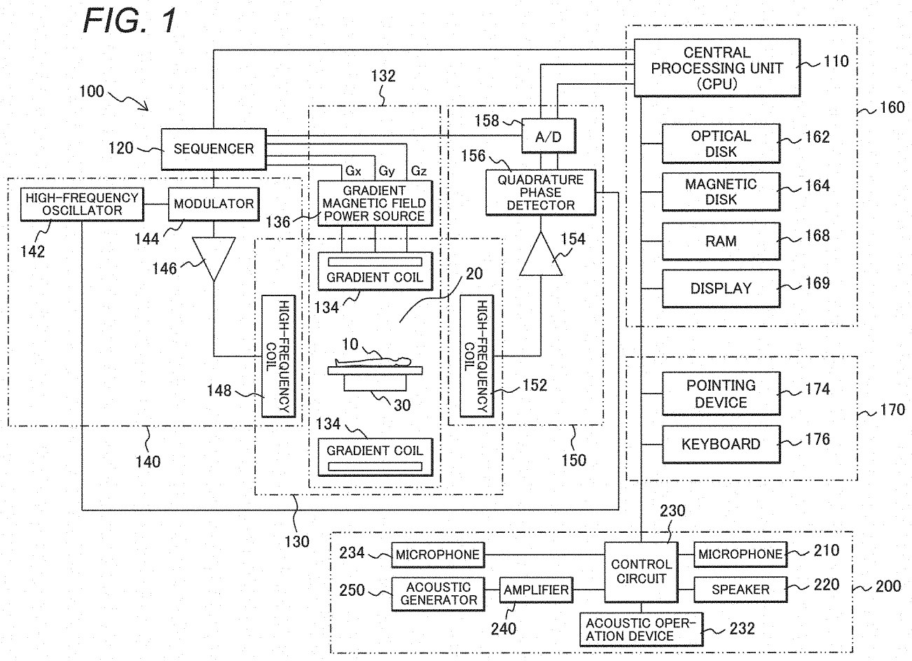

An embodiment of an MRI device 100 to which the present invention is applied will be described with reference to FIG. 1.

The MRI device 100 captures, for example, a tomographic image of a subject by using the NMR phenomenon and has a magnetostatic field generation device 130, a gradient magnetic field generation device 132, an RF signal irradiation device 140 emitting a high-frequency magnetic field pulse (hereinbelow, described as RF pulse), an NMR signal reception device 150 receiving an NMR signal as an echo signal, a processing device 160 having a central processing unit (hereinbelow, described as CPU) 110, a sequencer 120, an operation device 170 performing various operations related to input of data, imaging, and the like, and an acoustic system 200. Although the magnetostatic field generation device 130 has a magnetostatic field generation coil such as, for example, a superconductive coil for generating a very strong static magnetic field and a magnetic member for improving uniformity of the static magnetic field, to avoid complication, the magnetostatic field generation coil and the magnetic member are not illustrated.

A subject 10 laid on a bed 30 is disposed so that at least a part as an object to be imaged in the subject 10 is positioned in a measurement space 20 for performing imaging of the subject 10 or the like. The magnetostatic field generation device 130 has, as described above, the function of generating a magnetic field which is very uniform and further, very strong in the measurement space 20, and generates a very uniform static magnetic field in a direction orthogonal to the body axis of the subject 10 in space around the subject 10 in the case of the perpendicular magnetic field method or in the body-axis direction in the case of the horizontal magnetic field method. The magnetostatic field generation device 130 has a magnetostatic field generation source of a permanent magnet type, a normal conduction type, or a superconductive type around the subject 10 to generate the static magnetic field and has, in the normal conduction type or the superconductive type, a magnetostatic field generation coil for generating a static magnetic field as described in the description of the embodiment illustrated in FIG. 1.

The gradient magnetic field generation device 132 has: gradient coils 134 wound in three-axis directions of X axis, Y axis, and Z axis as the coordinate system, for example, a static coordinate system of the MRI device 100; and a gradient magnetic field power source 136 supplying drive current for generating a gradient magnetic field to each of the gradient coils. By operating the gradient magnetic field power source 136 in accordance with an instruction from the sequencer 120 which will be described later, drive current is supplied to the gradient coils 134 in the three-axis directions of the X axis, the Y axis, and the Z axis, and gradient magnetic fields Gx, Gy, and Gz in the three-axis directions of the X axis, the Y axis, and the Z axis are generated and applied to the part to be imaged in the subject 10. For example, at the time of imaging, a slice-direction gradient magnetic field pulse (Gs) is applied in a direction orthogonal to a slice plane as an imaging section to set a slice plane for the subject 10, a phase encode direction gradient magnetic field pulse (Gp) and a frequency encode direction gradient magnetic field pulse (Gf) are applied in the remaining two directions orthogonal to the slice plane and orthogonal to each other, and the position information in each of the directions is encoded in an NMR signal as an echo signal.

The sequencer 120 has the function of performing control of repetitively applying the RF pulse and the gradient magnetic field pulse in a predetermined pulse sequence in accordance with imaging schedule which is set, operates on the basis of a control instruction from the processing unit 110, and sends various control signals necessary for data collection of a section image of the subject 10 to devices which need the signals such as, for example, the RF signal irradiation device 140, the gradient magnetic field generation device 132, and the NMR signal reception device 150.

The RF signal irradiation device 140 has the function of irradiating the subject 10 with an RF pulse to make the atomic nucleus spins of atoms constructing a body tissue in the subject 10 cause nuclear magnetic resonance and has, for example, a high-frequency oscillator 142, a modulator 144, a high-frequency amplifier 146, and a high-frequency coil 148 on the transmission side operating as a transmission coil. A high-frequency pulse output from the high-frequency oscillator 142 is subject to amplitude modulation by the modulator 144 at a timing according to an instruction from the sequencer 120. By amplifying the amplitude-modulated high-frequency pulse by the high-frequency amplifier 146 and supplying the amplified RF pulse to the high-frequency coil 148 disposed close to the subject 10, the RF pulse is emitted to the subject 10.

The NMR signal reception device 150 has the function of detecting and processing an NMR signal as an echo signal emitted by the nuclear magnetic resonance of atomic nucleus spins constructing a body tissue in the subject 10 and has a high-frequency coil 152 on the reception side operating as a reception coil, a signal amplifier 154 amplifying a received NMR signal, a quadrature phase detector 156, and an A/D converter 158 converting an analog signal to a digital signal. An NMR signal as a response is generated from a tissue in the subject 10 induced by an RF pulse as an electromagnetic wave emitted from the high-frequency coil 148 on the transmission side, and the NMR signal is detected by the high-frequency coil 152 disposed close to the subject 10 and amplified by the signal amplifier 154. After that, the resultant signal is divided to signals of two orthogonal systems by the quadrature phase detector 156 at a timing according to an instruction from the sequencer 120. Each of the signals is converted to a digital amount by the A/D converter 158, and the resultant is transmitted to the processing device 160.

The processing device 160 has the function of performing various data processes, displaying and storing process results, and the like, and has external storage devices such as an optical disk 162 and a magnetic disk 164 for storing information, a RAM 168 performing temporal storage for process, and a display 169 such as a CRT. When a process result received and processed by the NMR signal reception device 150 is supplied from the NMR signal reception device 150 to the processing device 160, a signal process and a process such as image reconstruction are performed by the central processing unit 110 in the processing device 160. A tomographic image of the subject 10 as the result is displayed in the display 169 and, as necessary, stored in the optical disk 162, the magnetic disk 164, or the like as the external storage device. Although not illustrated, the tomographic image can also be printed or transmitted to another system.

The operation device 170 has the function of inputting various control information of the MRI device 100 and control information of processes performed by the processing device 160, and has a pointing device 174 such as a track ball or a mouse and a keyboard 176. The operation device 170 is disposed close to the display 169, and the operator can perform an operation for controlling various processes of the MRI device interactively via the operation device 170 while seeing display in the display 169. The operation device 170 is not limited to the above but may include, for example, a touch panel provided in the display plane of the display 169. The operation device 170 is provided in an operation room apart from the body of the MRI device 100 and, although not illustrated, in addition, a part of the operation device 170 is provided in the body of the MRI device 100 and the bed 30 and configured so that the operator can perform a necessary operation near the subject 10.

The high-frequency coil 148 and the gradient coil 134 on the transmission side are mounted so as to be opposed to the subject 10 in the perpendicular magnetic field method and so as to surround the subject 10 in the horizontal magnetic field method in the magnetostatic field space of the magnetostatic field generation device 130 in which the subject 10 is disposed. The high-frequency coil 152 on the reception side is mounted so as to be opposed to or surround the subject 10.

A nuclide to be imaged of the subject 10 is, for example, as a nuclide which is widely spread in clinical use, a hydrogen nucleus, that is, proton as a main component material of the subject. By imaging information regarding a space distribution of proton density and a space distribution of relaxation time in an excitation state, the shape or function of, for example, the head, abdomen, or extremities of the subject 10 is imaged two-dimensionally or three-dimensionally, and an obtained image is displayed in the display 169 or, as necessary, stored in the optical disk 162 or the magnetic disk 164, and printed, or transmitted to another necessary system on the basis of an operation.

### 3. Acoustic System 200

Communication between the operator and the subject 10 is necessary at the time of imaging the subject 10, so that the acoustic system 200 is provided. Further, the acoustic system 200 is not only for the communication but also can play music to make the subject 10 being imaged relaxed. Further, the acoustic system 200 also has the function of reducing, for example, noise generated by the gradient coil 134 on the basis of an operation. The acoustic system 200 has an acoustic control circuit 230, a microphone 210 on the operator side, a speaker 220, a microphone 234, and an acoustic generator 250 on the operator side, and an acoustic operation device 232 performing an operation related to the acoustic system 200.

The acoustic system 200 can adjust an output of the speaker 220 and the acoustic generator 250 by operating the acoustic operation device 232 and, in addition, has the function of outputting predetermined music from the acoustic generator 250 with a predetermined volume by operation of the acoustic control circuit 230 according to a control instruction from the central processing unit 110 and generating sound for cancelling out noise generated by the gradient coil 134 with a predetermined volume at a predetermined timing.

The speaker 220 is disposed in the operation room (not illustrated) and, although not illustrated, the speaker 220 has a structure of a general speaker and has, for example, a permanent magnet having a gap, a voice coil disposed in the gap, and a diaphragm which oscillates according to the movement of the voice coil. By interaction between a magnetic flux generated by the permanent magnet and current flowing in the voice coil, the voice coil oscillates according to the current, the diaphragm oscillates by the voice coil, and a sound is generated from the diaphragm. The microphone 234 and the acoustic generator 250 disposed on the side of the subject 10 are, preferably, disposed close to the subject 10 but may be disposed on the outside of the measurement space 20 so as not to hinder imaging and the like. Voice uttered by the subject 10 is converted to an electric signal by the microphone 234, and the electric signal is output from the speaker 220. On the other hand, voice of the operator is converted by the microphone 210 on the operator side to an electric signal. The electric signal is amplified by the amplifier 240 and converted to voice by the acoustic generator 250, and the voice is output. The acoustic generator 250 has a configuration different from that of the speaker 220 and has, for example, a structure of using a leakage flux passing the outside of the measurement space 20. The acoustic generator 250 uses the leakage flux passing the outside of the measurement space 20, thereby producing effects that disturbance of the magnetic field in the measurement space 20 by the acoustic generator 250 is suppressed and an adverse effect to the imaging operation of the MRI device 100, particularly, an adverse effect regarding deterioration in the picture quality can be suppressed. However, the present invention is not limited to use of the leakage flux passing the outside of the measurement space 20. The static magnetic field passing through the measurement space 20 may be used.

### 4. Configuration of Acoustic Generator 250 of Acoustic System 200

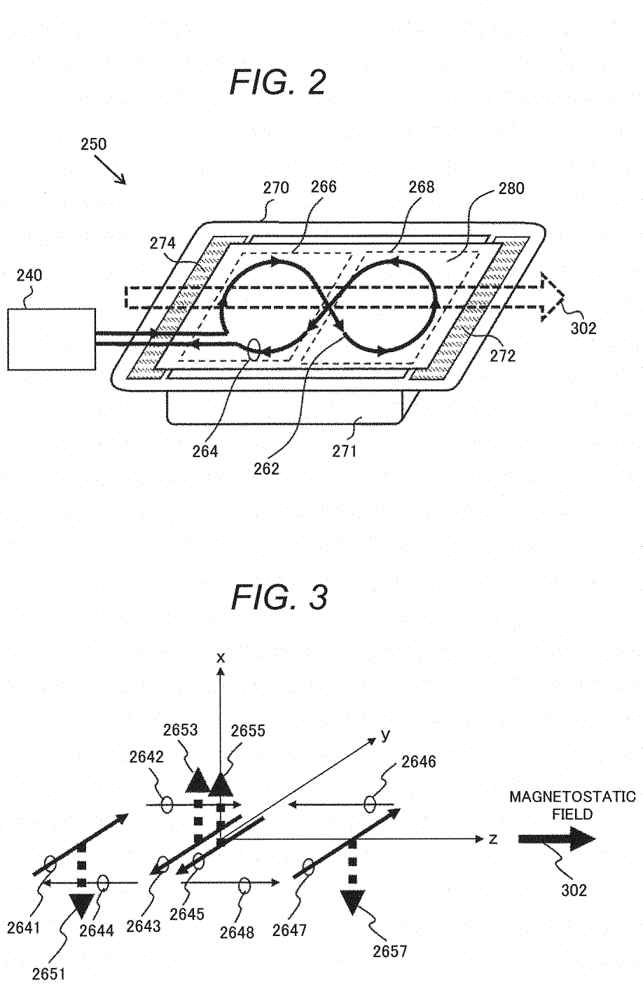

FIG. 2 is an explanatory diagram illustrating an embodiment of the acoustic generator 250 to which the present invention is applied. A diaphragm 280 having a thin shape and made of resin material is provided with an acoustic coil 262 having a figure-of-eight shape. In the embodiment, it is assumed that a magnetic field 302 from the MRI device 100 exists in the direction indicated by the arrow. Although the magnetic field 302 is illustrated only in a part of the diaphragm 280 to avoid complication, the intensities of the magnetic fields 302 are almost the same in the entire diaphragm 280, in other words, in the entire range of the acoustic coil 262.

The acoustic coil 262 has a first winding circuit 266 and a second winding circuit 268 which are wound in directions opposite to each other. In the embodiment, the first and second winding circuits 266 and 268 are connected in series. Since the first and second winding circuits 266 and 268 are connected in series, currents of the same value flow in the first and second winding circuits 266 and 268. Consequently, there is an effect that control is easy. However, also when the first and second winding circuits 266 and 268 are connected in parallel, they operate. In the embodiment illustrated in FIG. 2, the first and second winding circuits 266 and 268 are disposed so as to be deviated from each other and hardly overlapped each other in the direction of the magnetic field 302. Desirably, the first and second winding circuits 266 and 268 are disposed so as not to be overlapped in the direction of the magnetic field 302. However, even when there is a partial overlap part, the circuits normally operate. As the principle of operation of the acoustic coil 262 will be described later, the first and second winding circuits 266 and 268 constructing the acoustic coil 262 may also be provided, for example, separately on the surface and the rear face of the diaphragm 280. In such a structure, even when the first and second winding circuits 266 and 268 are partially overlapped or disposed apart from each other with a predetermined interval, they can operate as the acoustic generator 250.

It is assumed that current 264 for generating a sound from the amplifier 240 is supplied to the acoustic coil 262 and, as an example, current in the direction indicated by the arrow illustrated along the acoustic coil 262 flows at a certain moment. The diaphragm 280 is oscillatably supported by a supporting frame 270 via dampers 272 and 274. The current 264 flowing in the acoustic coil 262 changes on the basis of the direction and magnitude of the current from the amplifier 240, and the magnitude and direction of a force generated between the current 264 and the magnetic field 302 change according to the change in the current 264. According to the change in the force, the diaphragm 280 oscillates and air in the vicinity is vibrated, thereby generating a sound based on the current from the amplifier 240.

The diaphragm 280 is supported by the supporting frame 270 via the dampers 272 and 274. The dampers 272 and 274 have, for example, the function of a damper, oscillatably support the diaphragm 280 and, further, have the function of attenuating the oscillation of the diaphragm 280. Since the dampers 272 and 274 have the attenuation characteristic, the oscillation of the diaphragm 280 generated on the basis of the current 264 and the magnetic field 302 is properly attenuated, and continuation of the oscillation more than necessary can be prevented. Consequently, the oscillation of the diaphragm 280 faithfully follows the change of the current 264, and the quality of the sound generated by the diaphragm 280 improves.

In the embodiment, to simplify the configuration of the acoustic generator 250, the diaphragm 280 performing the operation of generating a sound is provided with the acoustic coil 262 passing the current 264 for generating a sound. The structure is very simple and has effects that productivity is excellent and a failure does not easily occur. However, the present invention is not limited to the structure. The acoustic coil 262 has a figure-of-eight shape and, as will be described later, has a structure that the force in the same direction is generated in the supporting part on the supporting member 272 side and the supporting part on the supporting member 274 side of the diaphragm 280. Consequently, torsion does not occur in the supporting parts of the diaphragm 280, and the movement of the diaphragm 280 can faithfully correspond to a change of the current 264. When the movement of the diaphragm 280 becomes complicated, there is a fear that the diaphragm 280 cannot faithfully correspond to a change of the current 264. In this viewpoint, in the embodiment, a change of the current 264 is easily reproduced in the displacement of the diaphragm 280 and, as a result, a sound of good quality can be generated. In other words, oscillation different from a change of the current 264 (that is, oscillation which deteriorates the sound quality) does not easily occur in the configuration. As described above, in the structure, the relation between the shape of the acoustic coil 262 illustrated in FIG. 2 and the supporting positions by the dampers 272 and 274 is suitable to improve the sound quality, and a very excellent effect that a sound of relatively good quality can be reproduced from small volume to large volume is produced. However, the present invention is not limited to the configuration. Another structure described later may also be employed. Even if the sound quality decreases a little or the characteristic deteriorates a little from the viewpoint of sound volume, the acoustic generator 250 having the characteristic improved more than that of a conventional one can be obtained.

### 4.1 Operation Principle of Acoustic Generator 250

With reference to FIGS. 3, 4, and 5, the operation principle of the acoustic generator 250 will be described. The acoustic coil 262 having a figure-of-eight shape can be considered that it is constructed by two acoustic coils in which currents flow in opposite directions and can be decomposed to eight current vectors as illustrated in FIG. 3. The eight current vectors are expressed as currents 2641, 2642, 2643, 2644, 2645, 2646, 2647, and 2648. The plane constructing the figure of eight is set as a YZ plane, the perpendicular direction is set as an X direction, and the vertical direction of the figure of “8” is set as a Z direction. In this case, in principle, the Lorentz force does not work on the currents in the same direction as the direction of the static magnetic field, that is, the currents 2642, 2644, 2646, and 2648. On the other hand, the Lorentz force works on the currents 2641, 2643, 2645, and 2647 perpendicular to the static magnetic field direction, force in the −X direction works on the currents 2641 and 2647, and force in the +X direction works on the currents 2643 and 2645.

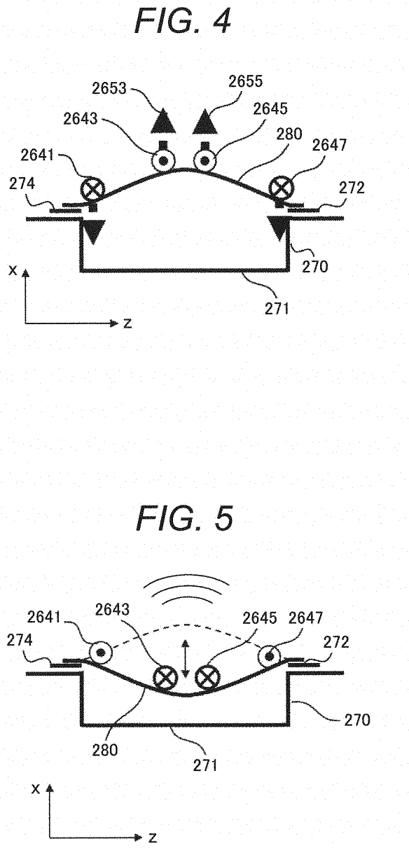

Sections of the XZ plane in FIG. 3 are illustrated in FIGS. 4 and 5. In FIG. 3, when the diaphragm 280 is fixed to the supporting frame 270, the currents 2643 and 2645 deform the diaphragm 280 in the direction of pulling it in the upward direction (+X direction). When it is assumed that current in the opposite direction flows, as illustrated in FIG. 5, the currents 2643 and 2645 generate force to deform the diaphragm 280 in the downward direction (−X direction). In such a manner, the currents 2643 and 2645 perform the operation of oscillating the diaphragm 280 in the perpendicular direction as the X-axis direction according to the direction of the flow as illustrated in FIG. 4 or 5. When the diaphragm 280 oscillates in the perpendicular direction, the air in the proximity of the diaphragm 280 is oscillated, and a sound based on the current 264 flowing in the acoustic coil 262 is generated.

Since the force of pulling and pushing in the Z direction works on the diaphragm 280 in the process of deformation of the diaphragm 280, to allow the displacement, the diaphragm 280 is supported by the supporting frame 270 via the dampers 272 and 274. The supporting frame 270 has a structure of covering one of the faces of the diaphragm 280. The primary role of the supporting frame 270 is the role as an exterior frame of the diaphragm. However, oscillation of the diaphragm causes swing of air on the both faces of the diaphragm. In some cases, the oscillation which comes around behind produces an unintended characteristic or the like of sound. To avoid it, as illustrated in FIGS. 2, 4, and 5, it is desirable for the supporting frame 270 to have a cover 271 to form a closed space.

As described above, by supporting the diaphragm 280 by the supporting frame 270 via the dampers 272 and 274, the operation of promptly attenuating the oscillation of the diaphragm 280 is performed and continuation of the oscillation more than necessary can be suppressed, so that the sound quality can be improved. Further, the diaphragm 280 itself is formed by a deformable resin substrate. A circuit pattern of the acoustic coil 262 can be formed in the diaphragm 280, and the function of generating sound is also provided. Like the dampers 272 and 274, the diaphragm 280 made by a resin substrate has a moderate oscillation attenuation characteristic, so that oscillation is not continued more than necessary, and can be properly attenuated. Consequently, excellent sound quality can be obtained. Further, as it is a resin substrate, there is an effect that an adverse influence on an MRI image captured by the MRI device 100 is not easily exerted.

### 4.2 Modification of Embodiment Illustrated in FIG. 2

In FIG. 2, placing importance on description of the operation principle, the acoustic generator 250 having a simplified configuration is described. As described above, the diaphragm 280 itself as the substrate provided with the acoustic coil 262 has the function of the diaphragm that generates a sound. However, the present invention is not limited to the structure.

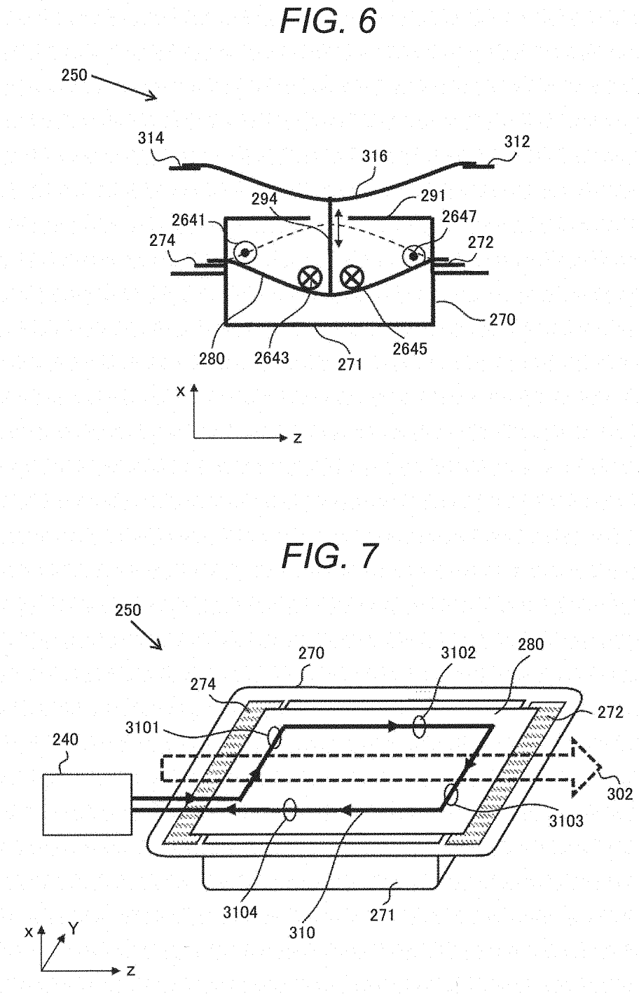

The acoustic generator 250 illustrated in FIG. 6 has a cone 316 for generating a sound, dampers 312 and 314 oscillatably supporting the cone 316, and an oscillation transmission mechanism 294 transmitting the oscillation of the diaphragm 280 to the cone 316. The diaphragm 280 is a substrate for providing the circuit of the acoustic coil 262. When the diaphragm 280 is oscillated too large, a problem occurs from the viewpoint of durability. Consequently, it is also possible to decrease the oscillation of the diaphragm 280 and produce a large sound by the cone 316. For example, by making the shape of the cone 316 larger than that of the diaphragm 280 and driving the cone 316 of a larger shape by the oscillation generated by the diaphragm 280, a larger sound can be generated. By using the diaphragm 280 as a drive source and providing the cone 316 separate from the diaphragm 280 as described above, an effect that the shape of the cone 316 can be made a shape suitable to a sound which is output is produced. When sounds are generated from both of the diaphragm 280 and the cone 316, there is the possibility that the sounds interfere each other. Consequently, it is preferable to provide the cover 271 and a cover 291 for covering the diaphragm 280.

### 4.3 Case of Forming Acoustic Coil 262 In One Loop Shape

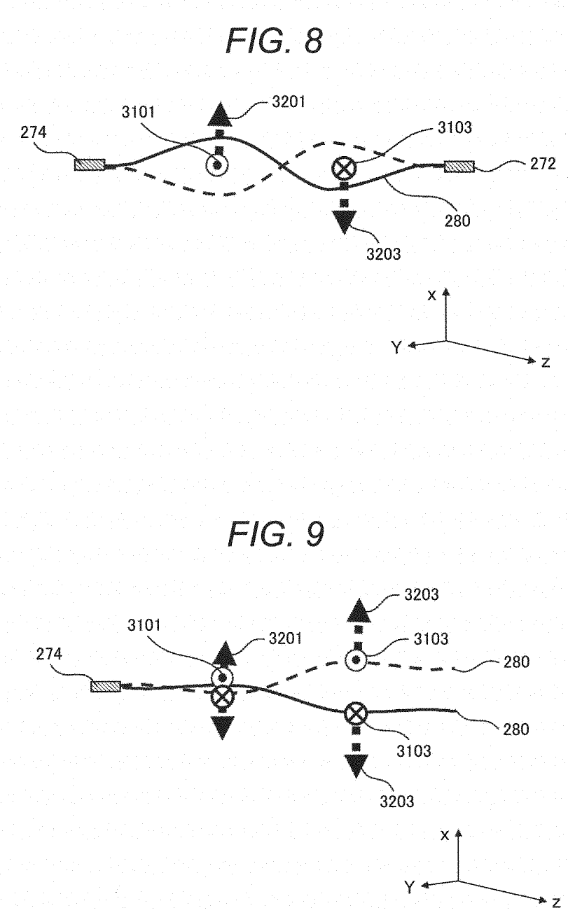

In FIG. 2, the acoustic coil 262 having the figure-of-eight shape (hereinbelow, described as figure-of-eight-shaped acoustic coil) is used. FIG. 7 illustrates an embodiment of using an acoustic coil 310 having a one loop shape in place of the figure-of-eight-shaped acoustic coil 262. The operation of the embodiment will be described with reference to FIG. 8. In FIG. 7, the acoustic coil 310 having a one loop shape is provided for the diaphragm 280. The one loop shape does not mean the number of turns of the acoustic coil. In FIG. 7, the shape of one turn is illustrated to describe the principle. However, the number of turns is not limited to one.

When the current flowing in the acoustic coil 310 is divided to current vectors in the X direction and the Y direction, it can be considered so as to be decomposed to currents 3101, 3102, 3103, and 3104. The currents 3102 and 3104 do not generate force in the relation with the direction of the magnetic field 302. On the other hand, the currents 3101 and 3103 operate with the magnetic field 302 and generate force. FIG. 8 illustrates the directions of forces generated by the currents 3101 and 3103. A force 3201 in the +X direction is generated by the current 3101, and a force 3203 in the −X direction is generated by the current 3103. Both ends of the diaphragm 280 in the Z-axis direction are supported by the dampers 272 and 274. Consequently, oscillation of the diaphragm 280 is small, and there is a problem that a sufficient sound cannot be generated.

FIG. 9 illustrates a state where the diaphragm 280 is supported by the damper 274 as one of the dumpers. The distance between the current 3103 and the damper 274 is longer than the positional relation between the current 3102 and the damper 274, and the diaphragm 280 can be largely oscillated by the force 3203 generated on the basis of the current 3103. By the structure, acoustics can be generated. In the embodiment of FIG. 9, since warpage itself of the diaphragm 280 does not contribute to generation of sound, the diaphragm 280 can be made of a relatively hard material. When the diaphragm 280 always deforms, durability becomes an issue at the time of forming a circuit on the diaphragm 280 which deforms. Since the warpage of the diaphragm 280, that is, periodical shape changes can be suppressed in the embodiment, the embodiment is excellent from the viewpoint of the durability of the acoustic coil 310. Although the diaphragm 280 is supported on the current 3101 side in the embodiment of FIG. 9, also in the case of supporting the diaphragm 280 by the damper 272 on the current 3103 side, the same action and effect can be obtained.

As another embodiment of FIG. 9, not generating acoustics directly by oscillation of the diaphragm 280 but a sound may be generated by the cone 316 by transmitting the oscillation of the diaphragm 280 to the cone 316 via the oscillation transmission mechanism 294 as described with reference to FIG. 6.

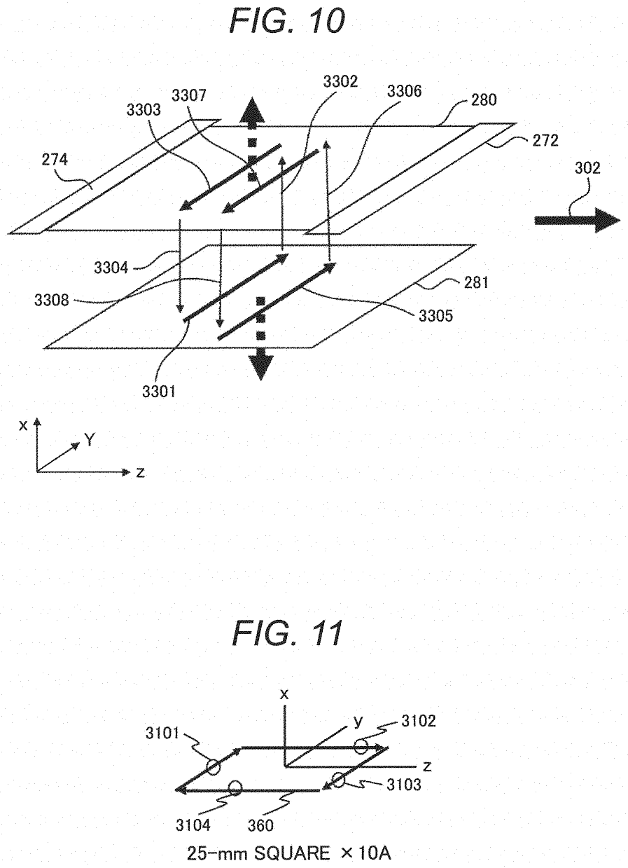

FIG. 10 is an explanatory diagram for explaining an embodiment of forming an acoustic coil in the Y-Z plane. Currents flowing in the acoustic coil will be described as separate currents 3301, 3302, 3303, 3304, 3305, 3306, 3307, and 3308. As illustrated, the currents 3301 and 3305 generate force in the −X direction, and the currents 3303 and 3307 generate force in the +X direction. Although not illustrated, the currents 3304 and 3308 generate force in the −Y direction, and the currents 3302 and 3306 generate force in the +Y direction. When a circuit passing the currents 3301 and 3305 is formed in a fixed substrate 281 and a circuit passing the currents 3303 and 3307 is formed in the diaphragm 280 which can displace in the X direction, the diaphragm 280 displaces according to the amount of the flowing current and the polarity of the current, and acoustics are generated. In this case, it is necessary to configure so that the length relations of the circuit passing the currents 3304 and 3308 and the circuit passing the currents 3302 and 3306 may change. In the embodiment, the diaphragm 280 is supported by the dampers 272 and 274 so that the diaphragm 280 can oscillate. In the embodiment, as an example, the structure that the circuit side in which the currents 3301 and 3305 flow is fixed and the circuit side in which the currents 3303 and 3307 flow can be fluctuate is employed. Alternatively, those relations can be made opposite. The diaphragm may be disposed not in the Y-Z plane but in the X-Z plane.

### Effects of Application to MRI Device 100

In the case of attachment to the MRI device 100, it is most important not to exert influence on MRI imaging. For example, in any of the foregoing embodiments, the static magnetic field generated by the magnetostatic field generation device 130 of the MRI device 100 is used. The static magnetic field is requested to have uniformity at extremely high precision. When the uniformity of the static magnetic field is lost, the quality of an image captured deteriorates. Based on this, the situation of generation of a magnetic flux from the acoustic coil in the case where current is supplied to the acoustic coil of the acoustic generator 250 is calculated.

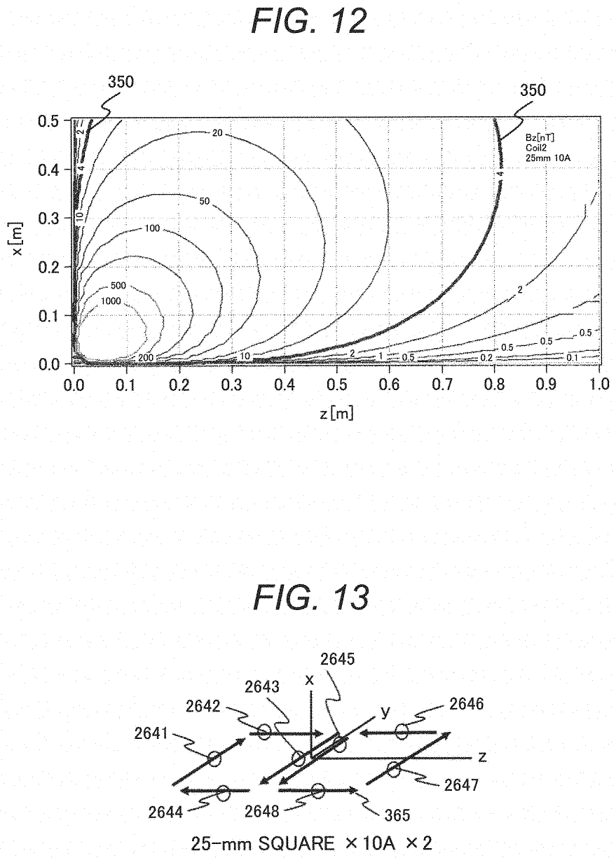

FIG. 11 illustrates the shape of the acoustic coil 360 used for calculation and current values supplied to the acoustic coil 360. FIG. 12 illustrates the relations between intensities of magnetic fields generated by the acoustic coil 360 as calculation results and distances. The acoustic coil 360 is an acoustic coil having a square shape whose one side is 25 mm and has one loop shape as illustrated in FIG. 7. The number of turns is one. The acoustic coil 360 is disposed in the Y-Z plane, and the current having the value of 10A is supplied to the acoustic coil 360. FIG. 12 illustrates the relation between the magnetic field strength and the distance in the X-Z plane. To clearly indicate the range of the magnetic field strength 4 nT, the border line 350 of the magnetic field strength 4 nT is indicated by a thick line. It can be regarded that the shorter the distance from the center of the acoustic coil 360 indicated by the border line 350 of 4 nT is, the smaller the influence exerted on the MRI device 100 is.

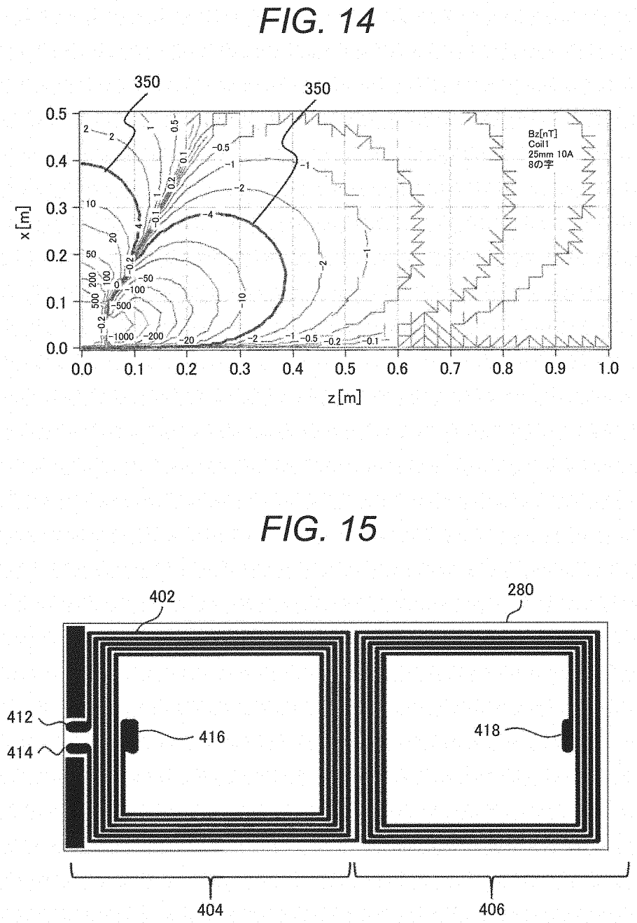

FIG. 13 illustrates the acoustic coil 365 having the figure-of-eight shape described with reference to FIG. 2. The acoustic coil 365 having the figure-of-eight shape is formed by combining two acoustic coils each of which has a square shape whose one side is 25 mm and which are wound in directions opposite to each other and deviated from each other in the Z-axis direction. The Z-axis direction is the direction of the static magnetic field. Like FIG. 11, the acoustic coil 365 having the figure-of-eight shape is disposed in the Y-Z plane, and current having the value of 10A is supplied to the acoustic coil 365 having the figure-of-eight shape. FIG. 14 illustrates the relations between the magnetic field intensities in the X-Z plane generated by the acoustic coil 365 having the figure-of-eight shape and distances. In the relations between the magnetic field intensities and the distances in FIG. 14, the border line 350 of the magnetic field strength 4 nT is indicated by a thick line.

When the calculation result illustrated in the graph of FIG. 12 and that illustrated in the graph of FIG. 14 are compared, the range of the magnetic field strength 4 nT in FIG. 14 is much narrower. It is therefore understood that by using the acoustic coil 365 having the figure-of-eight shape, the influence on the MRI device 100 can be largely reduced. As described also with reference to FIG. 2, the acoustic coil 365 having the figure-of-eight shape illustrated in FIG. 13 has two acoustic coils whose winding directions are opposite to each other and, moreover, the two acoustic coils generate magnetic fluxes of opposite polarities to current supplied from the amplifier 240. In other words, one of the acoustic coils emits the magnetic flux to the current supplied to the acoustic coil 365 having the figure-of-eight shape, and the other acoustic coil sucks the magnetic flux. Consequently, in the acoustic generator 250 as a whole, the general magnetic fluxes generated by the acoustic coil 365 having the figure-of-eight shape are cancelled off, and the influence on the other is extremely suppressed. Therefore, in the acoustic generator 250 using the static magnetic field of the MRI device 100, it is very preferable to use the acoustic coil 365 having the figure-of-eight shape to reduce the influence on imaging.

### 6. Configuration and Production Method of Acoustic Coil

In each of the foregoing embodiments, the configuration of the acoustic generator 250 including the acoustic coil has been simply described to explain the principle. Particularly, the acoustic coil as an important component will be described more specifically below. Although the acoustic coil having the figure-of-eight shape will be described as a representative example, a similar idea can be applied also to acoustic coils having other shapes. Although the acoustic coil having the figure-of-eight shape and whose number of turns is one is illustrated to avoid complication of the drawing, in reality, an acoustic coil having a concentric circle shape made of a plurality of turns and having proper impedance to the amplifier is desirable. For many audio amplifiers, impedance of 4 Ω to 8 Ω is appropriate. By properly selecting the number of turns of the acoustic coil, the impedance can be set to a proper value.

Although there are some methods of forming the acoustic coil, as an example, the acoustic coil can be formed by leaving a conductor 402 forming the acoustic coil by the technique of etching from a substrate having a structure in which copper foil is adhered to both faces of a polyimide film and removing unnecessary copper foil. By the conductor 402 left, an acoustic coil pattern is formed. By forming the acoustic coil pattern in such a manner, the productivity improves and high quality can be assured.

FIG. 15 illustrates an example of the acoustic coil pattern. In FIG. 15, only one of both faces is illustrated. However, the basis structure is the same. Connection terminals 412 and 414 are connected to the output terminal of an amplifier 240, and current for generating a sound is supplied from the amplifier 240 to the connection terminals 412 and 414.

The current supplied to the connection terminal 412 goes clockwise around a loop 404 on the left side in FIG. 15 and reaches a connection part 416 to the rear face on the inner side. The current moves to a pattern on the rear face, similarly goes clockwise, and shifts to the loop on the right side on the rear face. The current goes around the loop counterclockwise on the right side of the rear face and appears at a connection part 418 in the surface on the right side via the connection part in the rear face on the right side. The current enters a loop 406 on the right side in the surface from the connection part 418, goes counterclockwise around the right loop, and comes back to the connection terminal 414. In such a manner, the acoustic coil having the figure-of-eight shape can be easily formed in a double-sided print substrate.

Since the diaphragm operates by the force generated in the acoustic coil, the lighter the weight is, the faster the oscillation speed becomes. That is, a larger pressure fluctuation of air can be caused. On the other hand, when strength is insufficient, the diaphragm cannot bear the oscillation and is broken. From the aspect of such a characteristic, a polyimide film described above is appropriate.

The diaphragm 280 having the loops 404 and 406 and further, not illustrated loops in the rear face is supported by the supporting frame 270 so as to be oscillatable via the dampers 272 and 274 as illustrated in FIG. 2. The cover 271 suppresses emission of a sound generated by the rear face of the diaphragm 280. To sufficiently suppress the emission of the sound, it is desirable to make a resin for forming the cover 271 thick and, further, to provide a sound absorption material for the purpose of sound absorption on the inside of the cover 271.

The MRI device 100 obtains an image by emitting electromagnetic waves of RF frequency according to the magnetic field strength. There is, however, the possibility that the acoustic coil having the figure-of-eight shape operates as an antenna, absorbs the electromagnetic wave, causes an unnecessary loss or heat generation, and changes the distribution of the electromagnetic wave. To avoid this, it is preferable to add a balun in a some midpoint in the acoustic coil having the figure-of-eight shape or a power feed point until the acoustic coil having the figure-of-eight shape. Since the higher the resonance frequency becomes, the shorter the electric length of resonance becomes, it is preferable to dispose a balun at a short interval.

FIG. 15 illustrates a concrete example of the embodiment. In the acoustic coil in FIG. 15, the coil length in the surface and rear face is about four meters. As an example, the thickness of copper foil is 25 μm, and the width of a pattern is 0.5 mm. When the resistivity of copper is 16.78 nΩ·m, the resistance of the acoustic coil having the shape described here becomes about 5 Ω which is a value adapted to an acoustic device.

It is preferable to adjust the impedance of the acoustic coil to become a proper value by changing the width of the pattern or changing the number of turns according to the thickness of copper foil used or, further, by connecting a resistor in series or in parallel to the acoustic coil pattern.

The drive force for generating a sound is proportional to the product of the number of turns “n” of the acoustic coil pattern and current I flowing in the acoustic coil pattern. The current I is determined by a value obtained by dividing output voltage E of the amplifier 240 supplying current to the acoustic coil by total resistance “R” of the acoustic coil. The total resistance “R” of the acoustic coil is obtained by the product of resistance “r” per turn and the number of turns “n”. As a result, the driving force becomes as (n·I)∝[(n·E)/R]∝[(n·E)/(n·r)∝(E/r). Consequently, to increase the drive force, it is desirable to increase the output voltage E of the amplifier 240 or decrease the resistance “r” per turn of the acoustic coil pattern.

Since the total resistance “R” of the acoustic coil is determined in a certain range from the viewpoint of setting the impedance of the acoustic coil to an impedance adapted to the amplifier 240, as a result, the total resistance “R” of the acoustic coil is maintained in a predetermined range. Since the total resistance “R” becomes the product of the number of turns “n” of the acoustic coil and the resistance “r” per turn, to satisfy a condition of maintaining the total resistance “R” within the predetermined range, it is desirable to form the acoustic coil so as to increase the number of turns “n” of the acoustic coil and decrease the resistance “r” per turn in the acoustic coil pattern.

In the embodiment illustrated in FIG. 15, each of the loops 404 and 406 and, further, the not-illustrated conductor pattern in the rear face of the diaphragm 280 has a square shape, not a circular shape. As already described above, force is generated by the current having the relation perpendicular to the magnetostatic field. Therefore, the loops 404 and 406 are formed so that current perpendicular to the magnetostatic field flows. The shape may be a circular shape. However, the loops 404 and 406 having a square shape are excellent to form a larger number of conductor patterns by maintaining insulating property and the like in a predetermined area.

### 7. Application of Acoustic Generator 250 to Circular-Shaped MRI Device

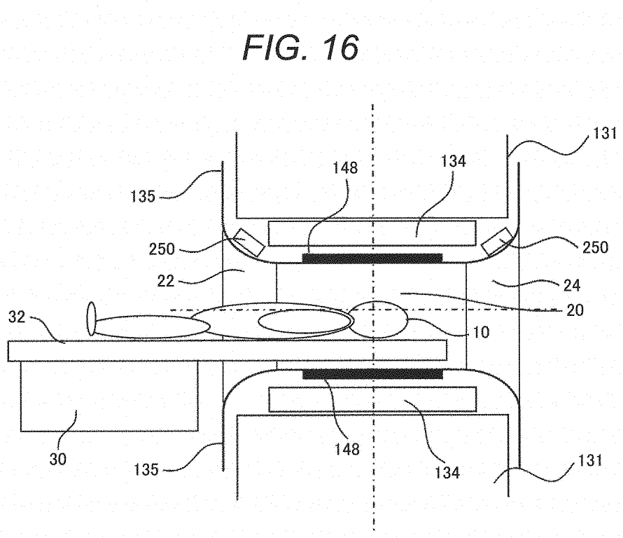

Disposition of the acoustic generator 250 in the MRI device 100 will now be described. FIG. 16 relates to an embodiment illustrating an example of disposing the acoustic generator 250 in an MRI device using a cylindrical magnet 131 in which a cylindrical space is formed. The cylindrical magnet 131 has, for example, a superconducting coil for generating a magnetostatic field and a cylindrical magnetic material having a cylindrical space in the center to uniform magnetic fields generated by the superconducting coil. On the side of the measurement space 20 of the cylindrical magnet, the gradient coil 134 and the high-frequency coil 148 are disposed. The cylindrical magnet 131 generates uniform magnetostatic field to the cylindrical measurement space 20, and the gradient coil 134 generates a gradient magnetic field. The subject 10 is sent by a top board 32 of the bed 30 so that a part to be imaged in the subject is positioned on the inside of the measurement space 20. The sequencer 120 generates a control signal on the basis of an instruction of the processing device 160 illustrated in FIG. 1, RF pulses are emitted from the high-frequency coil 148, and MRI imaging operation is performed.

Although dimensional margin is small in the proximity of the gradient coil 134 and the high-frequency coil 148, the acoustic generator 250 can be easily disposed in the proximity of both ends of the cylindrical space as opening parts 22 and 24 deviated in the body axis direction from the measurement space 20. It is therefore preferable to dispose the acoustic generator 250 on the inside of a cover 135 forming the opening parts 22 and 24. If it is on the inside of the cover 135 of the opening parts 22 and 24, there is a merit that disturbance of uniformity of the magnetostatic field of the measurement space 20 caused by the disposition of the acoustic generator 250 does not easily occur. In this case, a hole through which sound passes may be opened in the cover 135 so that sound from the acoustic generator 250 transmits easily to the subject 10. Alternatively, a displacement of the diaphragm 280 of the acoustic generator 250 may be transmitted to the cover 135 and sound is generated from the cover 135. In this case, to obtain sufficient sound pressure, by thinning the entire or part of the cover, a more preferable state is obtained. In the MRI imaging, various parts are imaged in various body postures. Therefore, the position of the head is not always constant and is not determined to exist in any of cylindrical bores, so that it is preferable to dispose the acoustic generator 250 at both ends of the cylinder. Both of the acoustic generators 250 disposed at both ends may be used or one of the acoustic generators 250 disposed at both ends may be selected and used on the basis of an operation from the acoustic operation device 232 and the operation device 170 illustrated in FIG. 1.

### Application of Acoustic Generator 250 to MRI Device of Perpendicular Magnetic Field Type

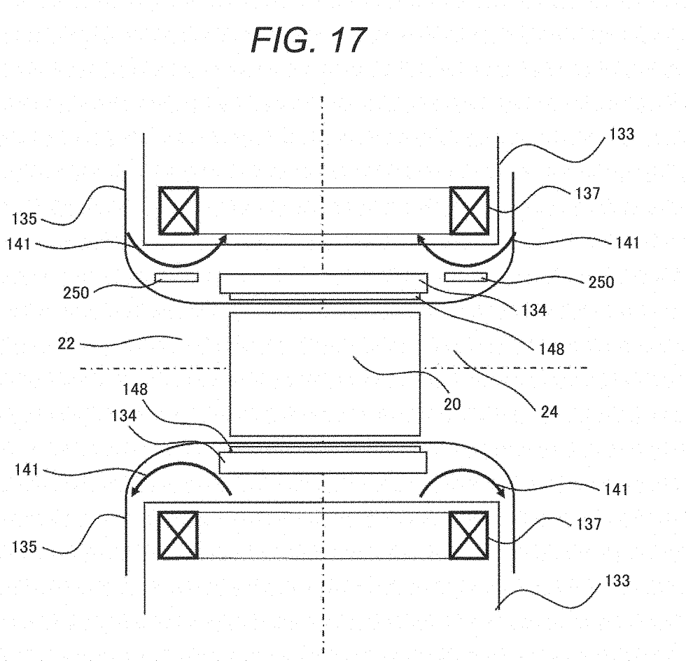

FIG. 17 illustrates an embodiment of disposing the acoustic generator 250 in an MRI device of a perpendicular magnetic field type. The MRI device of the perpendicular magnetic field type is an MRI device in which two disc-shaped magnetostatic field generation devices 133 are provided so as to sandwich the measurement space 20 in the vertical direction and a magnetostatic field in the vertical direction is generated in the measurement space 20 by the principle of the Helmholtz coil to perform MRI imaging. Further, the gradient coils 134 are disposed so as to sandwich the measurement space 20 and, further, the high-frequency coils 148 are disposed. Those are covered with the covers 135. The measurement space 20 in the MRI device of the perpendicular magnetic field type is relatively wide space. Further, the opening parts 22 and 24 are wider, and a space for disposing the acoustic generator 250 exists in the cover 135 forming the opening parts 22 and 24.

In the acoustic generator 250 whose principle is described with reference to FIG. 2, a plane part in which the acoustic coil 262 is disposed is disposed so as to be almost parallel to the magnetostatic field. That is, a component of the magnetostatic field almost parallel to the plane part acts with current flowing in the acoustic coil 262 to generate force for generating acoustic. In the measurement space 20 as the magnetic field center as illustrated, the magnetostatic field is in the vertical direction. In the magnet of the perpendicular magnetic field type, many superconducting coils 137 are wound at the end parts as illustrated, and the magnetic field generated by the magnetostatic field coil of stable direction and strength exits in the end parts. The direction of a magnetic flux 141 in the proximity is almost the horizontal direction. By using such a magnetic field, for example, only by disposing the acoustic generator 250 at the end part of the magnetic field generation source, sound of good quality can be generated. By disposing the acoustic generator 250 so that not only the part in the horizontal direction of the magnetic flux 141 but also the relation with the magnetic flux 141 become proper, the acoustic of good quality can be generated. If necessary, the perpendicular magnetic field generated by the superconducting coil 137 for generating magnetostatic field is used, and the acoustic generator 250 is disposed so as to have a predetermined relation with the perpendicular magnetic field, so that the acoustic can be generated with good quality.

The embodiment of FIG. 17 illustrates an executable example. By using the magnetic flux 141 of the superconducting coil 137, the acoustic generator 250 is disposed in the opening part 22 and/or the opening part 24. In the embodiment, the direction and strength of the magnetic flux 141 generated by the superconducting coil 137 are stable, so that the magnetic flux 141 is optimum to be used as the magnetic flux of the acoustic generator 250.

By being apart from the measurement space 20 only by predetermined distance, the possibility of disturbing the uniformity of the magnetostatic field in the measurement space 20 can be remarkably reduced. Further, the position is close to the head of the subject 10 and is suitable for communication with the subject 10.

### 9. Embodiment of Disposing Acoustic Generator 250 Using Blast Pipe for Cooling

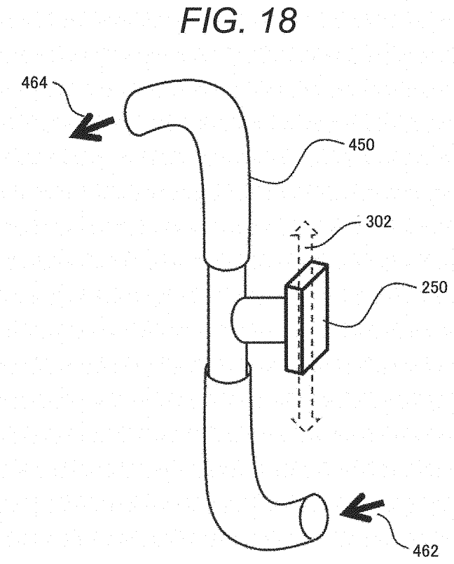

FIG. 18 illustrates an embodiment of disposing the acoustic generator 250 by using a blast pipe for cooling. Since a person as the subject 10 feels hot in the MRI device 10, a device for sending air to the person in the device via a blast pipe is provided. There is also a case of sending cooling air in order to maintain the temperature of the gradient coil 134, the high-frequency coil 148, or the like provided in a gantry as a part which performs imaging of the MRI device 100 to be constant and perform stable operation. When pressure fluctuation from the acoustic generator 250 is transmitted to the air flowing in such a blast pipe 450, the pressure fluctuation is diffused as sound in the opening of the blast pipe 450 to the person as the subject 10 or at the end part to which cooling wind of an irradiation coil or the like, and sound can be transmitted to the subject in the magnetostatic field generation source. In FIG. 18, the air 462 is taken in the blast pipe 450, and subject to pressure fluctuation by the acoustic generator 250 provided in some midpoint of the blast pipe 450. When the air 462 to which the pressure fluctuation is given is released from the blast pipe 450, sound is diffused. In the embodiment, even when the position of disposing the acoustic generator 250 is apart from the subject 10, sound can be transmitted via the blast tube 450. The acoustic generator 250 can be disposed in a position apart from the measurement space 20, and the influence on imaging can be suppressed. Since the blast pipe 450 passes near the magnetostatic field generation device 130, the acoustic generator 250 can be disposed in a proper position in the blast pipe 450 in consideration of the magnetostatic field. The magnetic field 302 in FIG. 18 is an example, and the magnetostatic field acts on the acoustic generator 250.

### 10. Use of Acoustic Generator 250 as Active Noise Canceller

The above-described acoustic generator 250 can generate air oscillation of good quality as a large output, which is difficult to be achieved by a conventional device. To cancel out noise generated by the gradient coil 134, the acoustic generator 250 capable of outputting large acoustic is necessary. The above-described acoustic generator 250 can do it and is used for such a purpose, thereby obtaining an effect which has not been obtained until now. As illustrated in FIG. 1, the central processing unit 110 transmits an instruction of current supply to the gradient coil 134 to the sequencer 120 and transmits a noise cancel instruction to the acoustic control circuit 230 in accordance with an imaging schedule. Based on the instruction, current having a wave shape instructed is supplied from the amplifier 240 to the acoustic generator 250 at an instructed timing. The acoustic generator 250 generates air vibration of the opposite phase for attenuating the noise generated by the gradient coil 134. As described above, large oscillation can be generated by the acoustic generator 250, and attenuation of noise generated by the gradient coil 134 which has not been able to be realized can be realized.

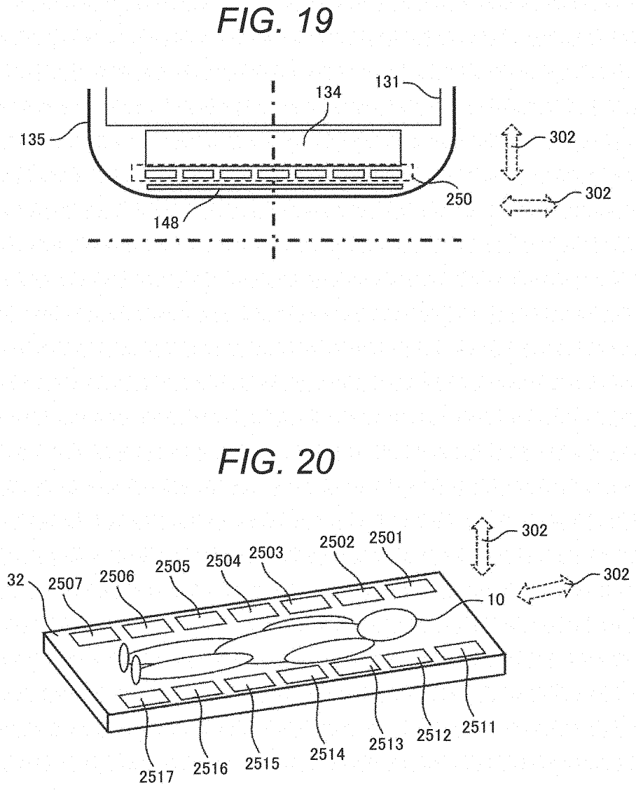

FIG. 19 illustrates an embodiment of disposing the acoustic generator 250 for performing active noise cancellation in a cylindrical magnet. In the case of performing active noise cancellation, first, the acoustic generator 250 is requested to generate volume equivalent to noise of an original gradient magnetic field.

Second, each of a plurality of acoustic generators 250 is controlled, and acoustic of the phase opposite to that of the noise of the gradient magnetic field is generated. To generate the acoustic having the relation of the phase opposite to that of the noise of the gradient magnetic field, it is preferable to accurately control the phase every position in which noise of the gradient magnetic field is transmitted. To adjust the phases more accurately, it is preferable to dispose and control a plurality of acoustic generators 250. FIG. 19 illustrates an example of disposing the acoustic generators 250 in line in a gap between the gradient coil 134 and the high-frequency coil 148. Although seven acoustic generators 250 are arranged, the invention is not limited to this number. By disposing the acoustic generators 250 so as to cover the surface of the gradient coil as the sound generation source as much as possible, noise can be cancelled effectively. Although not illustrated, it is most effective to arrange the acoustic generators 250 without a gap also in the angle direction. As excessive capability is unnecessary, the number of the acoustic generators 250 may be reduced in accordance with noise generated. Those controls can be performed by the acoustic control circuit 230 on the basis of an instruction from the central processing unit 110. Although one acoustic generator 250 is representatively illustrated in FIG. 1, the number of the acoustic generator is not limited to one. It is also possible to provide a number of acoustic generators 250 and the acoustic generators 250 of the number to be used can be selected as necessary.

To the acoustic generators 250 arranged, the magnetostatic field generated by the magnetostatic field generation device 130 is applied. The direction of the magnetic field 302 may be the body axis direction of the subject 10 or the vertical direction. Each of the acoustic generators 250 is fixed so that acoustic coils of the acoustic generators 250 are disposed so as to be aligned to the direction of the magnetostatic field.

A plurality of acoustic generators 250 may be disposed in the top board 32 of the bed 30. FIG. 12 illustrates an embodiment in the case of disposing a number of acoustic generators 250 in the top board 32 on which a subject is laid. Disposition of the acoustic generators 250 to the top board 32 has two meanings. In one meaning, when acoustic generators 250 are arranged like acoustic generators 2501 to 2507 and acoustic generators 2511 to 2517 as illustrated in FIG. 20, even when the acoustic generator 250 is disposed on the bottom side of the subject 10, that is, on the lower side of the top board, it is difficult to perform control of reducing noise on the subject side because of interruption of the top board having rigidity. This situation can be improved by using the acoustic generator 250. In another meaning, combinations of relative position relation between the top board and the subject are overwhelmingly smaller as compared with combinations of position relations between a gantry and a subject. Specifically, when a transducer is disposed in the top board, the place in which sound is to be reduced can be easily specified. Therefore, precision of reduction of noise improves. The magnetic field 302 is applied in the body axis direction of the subject 10 or the vertical direction with respect to the subject 10 to the top board 32, and the acoustic generators 2501 to 2507 and the acoustic generators 2511 to 2517 are provided so as to form a predetermined angle with the magnetic field 302.

In the above description, when the magnetic field 302 and the acoustic coil 262 of the acoustic generators 250 have the perpendicular relation, force is generated by the action between the current flowing in the acoustic coil 262 and the magnetic field 302. However, all of the magnetic fields 302 do not have to have the perpendicular relation to the acoustic coil 262. When the magnetic field 302 has a component having a perpendicular relation with the acoustic coil 262, force is generated between the current flowing in the acoustic coil 262 and the component of the magnetic field 302 having the perpendicular relation. Although the magnetic field 302 is described in the above description, not only all of the magnetic field 302 but the component may be sufficient.

### REFERENCE SIGNS LIST

10 . . . subject,
20 . . . measurement space,
22 . . . opening part,
24 . . . opening part,
30 . . . bed,
32 . . . top board,
100 . . . MRI device,
110 . . . central processing device,
120 . . . sequencer,
130 . . . magnetostatic generation device,
132 . . . gradient magnetic field generation device,
134 . . . gradient coil
135 . . . cover,
136 . . . gradient magnetic field power source,
140 . . . RF signal irradiation device,
142 . . . high-frequency oscillator,
144 . . . modulator,
146 . . . high-frequency amplifier,
148 . . . high-frequency coil,
150 . . . NMR signal reception device,
160 . . . processing device,
162 . . . optical disk,
164 . . . magnetic disk,
166 . . . ROM,
168 . . . RAM,
169 . . . display,
170 . . . operation device,
174 . . . pointing device,
176 . . . keyboard,
200 . . . acoustic system,
210 . . . microphone,
220 . . . speaker,
230 . . . acoustic control circuit,
232 . . . acoustic operation device,
234 . . . microphone,
240 . . . amplifier,
250 . . . acoustic generator,
262 . . . acoustic coil,
264 . . . current,
270 . . . supporting frame,
271 . . . cover,
272 . . . damper,
274 . . . damper,
280 . . . diaphragm,
302 . . . magnetic field,
310 . . . acoustic coil,
404 . . . loop,
406 . . . loop,
450 . . . blast pipe

1. An acoustic generator for an MRI device, comprising:
an acoustic coil provided in a magnetic field for imaging generated by a magnetostatic field generation coil or a gradient magnetic field generation coil of an MRI device or in a magnetic field for imaging generated by both of the magnetostatic field generation coil and the gradient magnetic field generation coil, separately from the magnetostatic field generation coil and the gradient magnetic field generation coil;
a diaphragm which oscillates on the basis of a force generated by the acoustic coil by action between current flowing in the acoustic coil and the magnetic field for imaging generated by the MRI device; and
a supporting member supporting the diaphragm so as to be oscillatable,
wherein when current whose value changes to generate acoustics flows in the acoustic coil, the force generated in the acoustic coil changes according to a change in the current, and the diaphragm vibrates air in accordance with the change in the force, thereby generating acoustics.

2. The acoustic generator for an MRI device according to claim 1, wherein the acoustic coil is provided for a resin substrate, and at least a part of a plane in which the acoustic coil is provided of the resin substrate is configured so as to be able to be in parallel to the magnetic field for imaging.

3. The acoustic generator for an MRI device according to claim 2, wherein the acoustic coil has a first winding circuit and a second winding circuit wound in directions opposite to each other, and the first and second winding circuits are disposed so as to be deviated from each other in a direction along the parallel component of the magnetic field.

4. The acoustic generator for an MRI device according to claim 3, wherein the first and second winding circuits are connected in series and provided for the resin substrate.

5. An MRI device comprising the acoustic generator for an MRI device according to claim 1, comprising:
a magnetostatic field generation device having the magnetostatic field generation coil generating a static magnetic field in a measurement space;
a gradient magnetic field generation device having the gradient magnetic field generation coil generating a gradient magnetic field in the measurement space;
a high-frequency coil emitting an RF pulse; an NMR signal reception device receiving and processing an NMR signal generated on the basis of emission of the RF pulse; and
a processing device reconstructing an image on the basis of a result of the process of the NMR signal reception device,
wherein the force is generated in the acoustic coil by the static magnetic field generated by the magnetostatic field generation device and current flowing in the acoustic coil of the acoustic generator for the MRI device, and
the diaphragm of the acoustic generator for the MRI device oscillates by the force to generate acoustics.

6. The MRI device according to claim 5, wherein a cover is provided and
the magnetostatic field generation device, the gradient magnetic field generation device, and the acoustic generator for the MRI device are provided on the inside of the cover.

7. The MRI device according to claim 5, wherein the acoustic generator for the MRI device is disposed on the inside of the cover forming an opening part positioned on the outside of the measurement space.

8. The MRI device according to claim 5, wherein a blast pipe for sending air is provided, the blast pipe is provided with the acoustic generator for the MRI device, and acoustics generated by the acoustic generator for the MRI device are transmitted via the blast pipe.

9. The MRI device according to claim 5, wherein the acoustic generator for the MRI device is disposed near the high-frequency coil emitting the RF pulse, force is generated by the static magnetic field generated by the magnetostatic field generation device in the acoustic coil of the acoustic generator for the MRI device, and the diaphragm generates acoustics by the force.

10. The MRI device according to claim 5, wherein the acoustic generator for the MRI device is disposed near the gradient coil generating the gradient magnetic field.

11. The MRI device according to claim 5, wherein the acoustic generator for the MRI device is disposed between the gradient coil generating the gradient magnetic field and the high-frequency coil emitting the RF pulse.

12. The MRI device according to claim 5, wherein a bed having a top board is further provided and the acoustic generator for the MRI device is disposed in the top board of the bed.

13. The MRI device according to claim 9, wherein a plurality of the acoustic generators for the MRI device are arranged, and a sound for reducing a sound generated by the gradient coil is generated by the acoustic generator for the MRI device.

14. The MRI device according to claim 5, further comprising a microphone on an operator side,
wherein a sound is converted to an electric signal by the microphone on the operator side, current based on the electric signal is supplied to the acoustic coil of the acoustic generator for the MRI device, and the acoustic based on the sound is output from the acoustic generator for the MRI device.
